(19) United States
(12) Patent Application Publication
(10) Pub. No.: US 2011/0152709 A1
(43) Pub. Date: Jun. 23, 2011

(54) MOBILE BODY CONTROL DEVICE AND MOBILE BODY CONTROL METHOD

(75) Inventors: Hitoshi Yamada, Aichi-gun (JP); Tomoya Takatani, Nisshin-shi (JP); Kazutoshi Ebe, Nisshin-shi (JP); Andrzej Cichocki, Wako-shi (JP); Kyuwan Choi, Wako-shi (JP)

(73) Assignees: TOYOTA JIDOSHA KABUSHIKI KAISHA, Toyota-shi (JP); RIKEN, Wako-shi (JP)

(21) Appl. No.: 13/060,082

(22) PCT Filed: Sep. 18, 2009
(86) PCT No.: PCT/JP2009/004749

§ 371 Date: Feb. 22, 2011

(30) Foreign Application Priority Data
Oct. 29, 2008 (JP) 2008-278348

(51) Int. Cl. A61B 5/0476

(52) U.S. Cl. 600/544

(57) ABSTRACT

A mobile body control device includes: a brain activity detecting unit that detects brain activity information of a user; a brain signal separating unit that separates an artifact component from the brain activity information detected by the brain activity detecting unit; a control signal generating unit that slides a sampling period for extracting brain data, at predetermined intervals in an overlapped manner in the brain activity information from which the artifact component is separated, successively calculates feature values for the brain data within each of the sampling periods obtained by sliding, and generates a control signal based on the feature values calculated; and a drive control unit that drives and controls a mobile body with a user riding thereon, based on the control signal generated.

### TECHNICAL FIELD

The present invention relates to a mobile body control device and a mobile body control method for controlling a mobile body that travels with a user riding thereon. In particular, the present invention relates to a mobile body control device and a mobile body control method for driving and controlling a mobile body based on brain activity information of a user.

### BACKGROUND ART

In recent years, various control methods have been proposed as a method for operating a mobile body by utilizing brain activity information of a user. A control utilizing this brain activity information has an advantage capable of providing swiftness, a user-friendly interface, or an interface that can also be used by a person with disabled limbs, for example. It is difficult to obtain such an advantage in conventional controls such as a control utilizing a myoelectric potential and a control utilizing an operation system such as a joystick.

Meanwhile, there is known an activity supporting system that drives and controls an electric wheelchair according to a degree of attention of a user based on a variation pattern of the brain wave intensity of the user, and an attention area in the field of vision of the user based on a brain wave intensity distribution and the line of sight (e.g., see Patent Literature 1). Further, a technique is disclosed in which an electric wheelchair to be controlled by brain waves is mounted with a variety of obstacle sensors, and the electric wheelchair is reliably controlled by utilizing sensor information from the obstacle sensors according to the needs of a user (e.g., see Non Patent Literature 1).

### Citation List

### Patent Literature

[Patent Literature 1] Japanese Unexamined Patent Application Publication No. 2007-202882

### Non Patent Literature

[Non Patent Literature 1] 2007 IEEE 10th International Conference (Adaptive Shared Control of a Brain-Actuated Simulated Wheelchair)

### SUMMARY OF INVENTION

### Technical Problem

Incidentally, brain activity information detected by sensors generally has a small signal/noise ratio. Accordingly, it is difficult to generate a control signal with sufficient accuracy by separating and extracting desired brain activity information. On the other hand, the activity supporting system disclosed in Patent Literature 1 drives and controls an electric wheelchair according to the degree of attention of the user and the attention area in the field of vision of the user.

However, the structure and control processing thereof are complicated. In the related art disclosed in Non Patent Literature 1, it seems to be possible to more reliably control the electric wheelchair by utilizing the sensor information from the obstacle sensors. However, the structure and control processing of the electric wheelchair are complicated.

The present invention has been made to solve the above-mentioned problem, and a principal object of the present invention is to provide a mobile body control device and a mobile body control method that are capable of achieving a highly accurate control while simplifying control processing.

### Solution to Problem

To achieve the above-mentioned object, one aspect of the present invention is a mobile body control device including: a brain activity detecting unit that detects brain activity information of a user; a brain signal separating unit that separates an artifact component from the brain activity information detected by the brain activity detecting unit; a control signal generating unit that slides a sampling period for extracting brain data, at predetermined intervals in an overlapped manner in the brain activity information from which the artifact component is separated by the brain signal separating unit, successively calculates feature values for the brain data within each of the sampling periods obtained by sliding, and generates a control signal based on the feature values calculated; and a drive control unit that drives and controls a mobile body with a user riding thereon, based on the control signal generated by the control signal generating unit. According to this aspect, it is possible to achieve a highly accurate control while simplifying control processing.

In this aspect, there may be further provided a teacher signal generating unit that generates a teacher signal, and the brain signal separating unit may perform learning using the teacher signal generated by the teacher signal generating unit, and may separate the artifact component depending on a user.

In this aspect, there may be further provided a teacher signal generating unit that generates a teacher signal, and the control signal generating unit may calculate a correlation between the feature values and the control signal by using the teacher signal generated by the teacher signal generating unit, and may generate the control signal based on the feature values and the correlation calculated.

In this aspect, there may be further provided a myoelectric potential detecting unit that detects a myoelectric potential of a user, and a stop determining unit that determines whether or not to stop the mobile body based on the myoelectric potential detected by the myoelectric potential detecting unit. When the stop determining unit determines to stop the mobile body, the drive control unit may control the mobile body to be stopped.

In this aspect, when the drive control unit successively receives a same control signal from the control signal generating unit a predetermined number of times or more, the drive control unit may execute a control corresponding to the control signal.

In this aspect, the control signal generating unit may include: a signal generating unit that successively generates control signals corresponding to the feature values based on the feature values calculated and on a preset correlation between the feature values and the control signals; and a signal selecting unit that divides the control signals successively generated by the signal generating unit into groups each having a predetermined number of successive control signals, selects at least one control signal from each of the groups, and outputs the selected control signal to the drive control unit.

In this aspect, the signal selecting unit may select, for each group, a control signal of a type which is largest in number in each of the groups, and may output the selected control signal to the drive control unit.

In this aspect, the signal selecting unit may form the groups each having a current control signal and successive previous control signals.

In this aspect, the control signal generating unit may generate the control signal based on the myoelectric potential detected by the myoelectric potential detecting unit, and when a control signal generated based on the myoelectric potential and a control signal generated based on the brain activity information are compared and when they are different from each other, the drive control unit may drive and control, or stop the mobile body according to the control signal generated based on the myoelectric potential.

Further, in this aspect, there may be further provided a myoelectric potential detecting unit that detects a myoelectric potential of a user. The control signal generating unit may be configured to be capable of generating the control signal based on the myoelectric potential detected by the myoelectric potential detecting unit. Additionally, when a control content based on the brain activity information is different from a control content based on the myoelectric potential, the control signal generating unit may supply the control signal indicating the control content based on the myoelectric potential to the drive control unit.

Furthermore, in this aspect, the control signal generating unit may sequentially make judgment using the feature values, and may determine one control content by majority among a plurality of results of judgment sequentially made. Then, the control signal generating unit may supply the control signal indicating the control content determined by the majority to the drive control unit.

Furthermore, in this aspect, the brain activity detecting unit may include at least three sensor groups. Here, each of the at least three sensor groups includes at least one sensor that detects a brain wave signal of a user. In this case, the control signal generating unit may determine one control content by majority when control contents based on the brain activity information detected by each of the sensor groups are different from each other, and may supply the control signal indicating the control content determined by the majority to the drive control unit. Additionally, in this aspect, there may be further provided perception means for allowing a user to perceive a control result based on the control signal generated by the control signal generating unit, and the perception means may be a visual-feedback unit that visualizes the control result.

On the other hand, to achieve the above-mentioned object, another aspect of the present invention may be a mobile body control method including: a brain activity detection step of detecting brain activity information of a user; a brain signal separation step of separating an artifact component from the brain activity information detected in the brain activity detection step; a feature value calculation step of sliding a sampling period for extracting brain data, at predetermined intervals in an overlapped manner in the brain activity information from which the artifact component is separated in the brain signal separating step, and successively calculating feature values for the brain data within each of the sampling periods obtained by sliding; a control signal generation step of generating a control signal based on the feature values calculated in the feature value calculation step; and a drive control step of driving and controlling a mobile body with the user riding thereon, based on the control signal generated in the control signal generation step.

### ADVANTAGEOUS EFFECTS OF INVENTION

According to the present invention, it is possible to achieve a highly accurate control while simplifying control processing in a mobile body control device and a mobile body control method.

### BRIEF DESCRIPTION OF DRAWINGS

FIG. 1 is a block diagram showing an example of a system configuration of a mobile body control device according to a first embodiment of the present invention;

FIG. 2 is a view showing the head of a user viewed from the above, and showing five electrodes arranged on the head;

FIG. 3A is a graph showing an example of brain data in which a sampling period in each brain wave signal is divided at regular small intervals;

FIG. 3B is a graph showing an example of a state in which a sampling period in a brain wave signal X is slid at each predetermined short time in an overlapped manner;

FIG. 4 is a flowchart showing an example of a control processing flow of the mobile body control device according to the first embodiment of the present invention;

FIG. 5 is a block diagram showing an example of a system configuration of a mobile body control device according to a second embodiment of the present invention;

FIG. 6 is a block diagram showing an example of a system configuration of a mobile body control device according to a third embodiment of the present invention;

FIG. 7 is a diagram showing an example of a selection method by a signal selecting unit of a control signal generating unit according to the third embodiment of the present invention;

FIG. 8 is a block diagram showing an example of a system configuration of a mobile body control device according to a fourth embodiment of the present invention; and

FIG. 9 is a block diagram showing an example of a system configuration of a mobile body control device according to a fifth embodiment of the present invention.

### DESCRIPTION OF EMBODIMENTS

Hereinafter, best modes for carrying out the present invention will be described by way of embodiments with reference to the accompanying drawings.

### First Embodiment

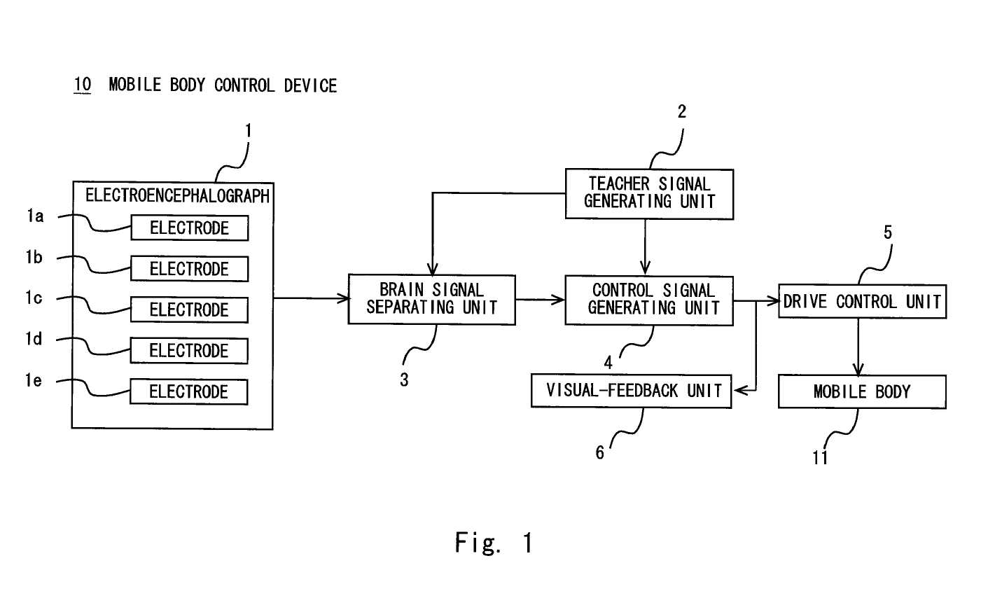

FIG. 1 is a block diagram showing an example of a system configuration of a mobile body control device according to a first embodiment of the present invention. A mobile body control device 10 according the first embodiment controls driving of a mobile body (e.g., electric wheelchair) 11 that moves with a user riding thereon. The mobile body control device 10 includes an electroencephalograph 1, a teacher signal generating unit 2, a brain signal separating unit 3, a control signal generating unit 4, a drive control unit 5, and a visual-feedback unit 6.

Note that the mobile body control device 10 is configured with a microcomputer as the center. The microcomputer includes, as main hardware components, a CPU (Central Processing Unit) that performs control processing, arithmetic processing, and the like, a ROM (Read Only Memory) that stores a control program, an arithmetic program, or the like executed by the CPU, and a RAM (Random Access Memory) that temporarily stores processing data or the like. The brain signal separating unit 3, the control signal generating unit 4, the drive control unit 5, the visual-feedback unit 6, and a stop determining unit 22, which is described later, are implemented by software which is stored in the ROM, for example, and is executed by the CPU.

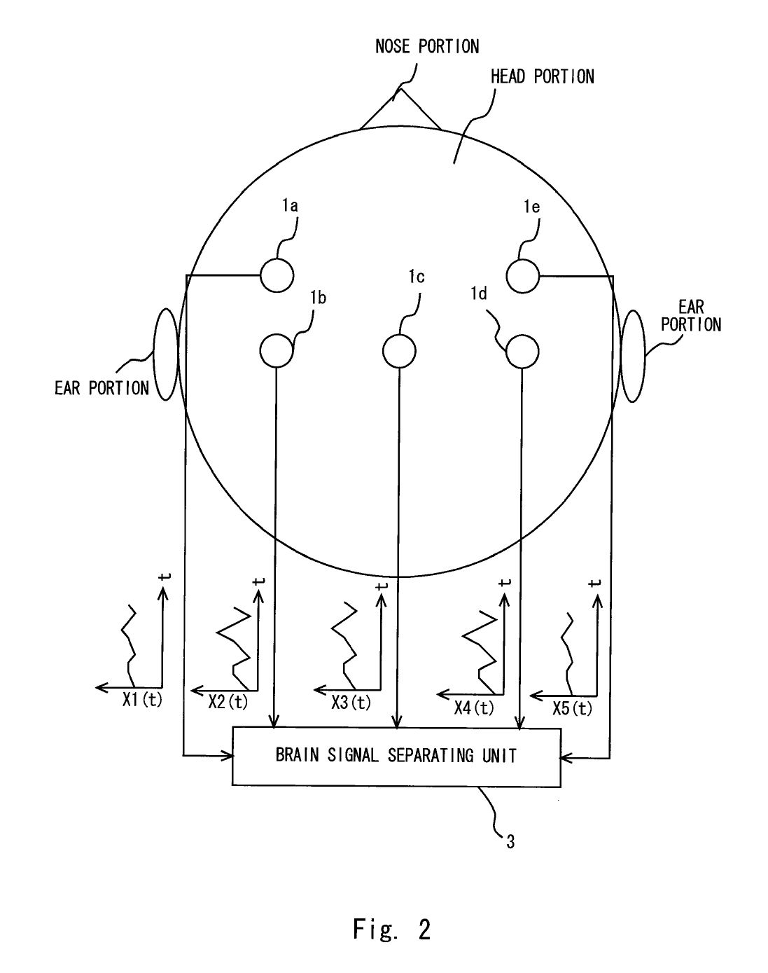

The electroencephalograph (brain activity detecting unit) 1 includes five electrodes 1a, 1b, 1c, 1d, and 1e which are arranged on the head of a user, for example (FIG. 2), and measures and detects brain activity information in the vicinity of the primary motor area of the head of the user. Each of electrodes 1a to 1e can detect brain wave signals, such as θ-wave (4 to 8 Hz), α-wave (8 to 12 Hz), and β-wave (12 to 40 Hz), as the brain activity information. The electrodes 1a to 1e of the electroencephalograph 1 output measured user's brain wave signals X1(t), X2(t), X3(t), X4(t), and X5(t) (“t” represents time), respectively, to the brain signal separating unit 3.

The teacher signal generating unit 2 generates a teacher signal for each of the brain signal separating unit 3 and the control signal generating unit 4, as described later. The teacher signal generating unit 2 includes a posture sensor such as a gyroscopic sensor or an acceleration sensor which can detect a posture value (a roll angle, a pitch angle, a yaw angle, or the like) of the head of the user. The teacher signal generating unit 2 generates the teacher signal based on the posture value of the user which is detected by the posture sensor.

The brain signal separating unit 3 first amplifies the brain wave signals from the electrodes 1a to 1e of the electroencephalograph 1, and converts the amplified signals into digital signals. Further, the brain signal separating unit 3 executes adaptive filtering processing for separating and removing an artifact component from each of the brain wave signals, which are amplified and converted into digital signals, by using a blind signal separation algorithm. Thus, noise signals (artifacts) induced by cardiac muscles, ocular muscles, or the like except for the brain activities are removed from the brain wave signals. This makes it possible to increase a brain wave signal/noise ratio and to detect a highly-accurate brain wave signal. Note that the blind signal separation algorithm is a well-known signal separation algorithm based on AMUSE method, so the detailed description thereof is omitted.

Furthermore, the brain signal separating unit 3 may learn in advance artifact components included in the brain wave signals of the user riding on the mobile body 11, by using a learning algorithm, such as a neural network, based on the teacher signal input from the teacher signal generating unit 2, and may construct an optimum filter for each user. As a result, the artifact components can be separated from the brain wave signals with high accuracy depending on the characteristics of each user. The brain signal separating unit 3 outputs the brain wave signals, from which the artifact components are separated, to the control signal generating unit 4.

The control signal generating unit 4 generates a control signal (e.g., a forward movement signal, a backward movement signal, a right turn signal, or a left turn signal) for driving and controlling (e.g., forward movement control, backward movement control, right turn control, or left turn control) the mobile body 11 based on the brain wave signals from the brain signal separating unit 3.

The control signal generating unit 4 successively calculates a feature value fp by a CSP method (common spatial patterns method) based on the brain wave signals X1(t), X2(t), X3(t), X4(t), and X5(t) which are composed of short time sequences. Then, the control signal generating unit 4 successively generates control signals based on the calculated feature value fp.

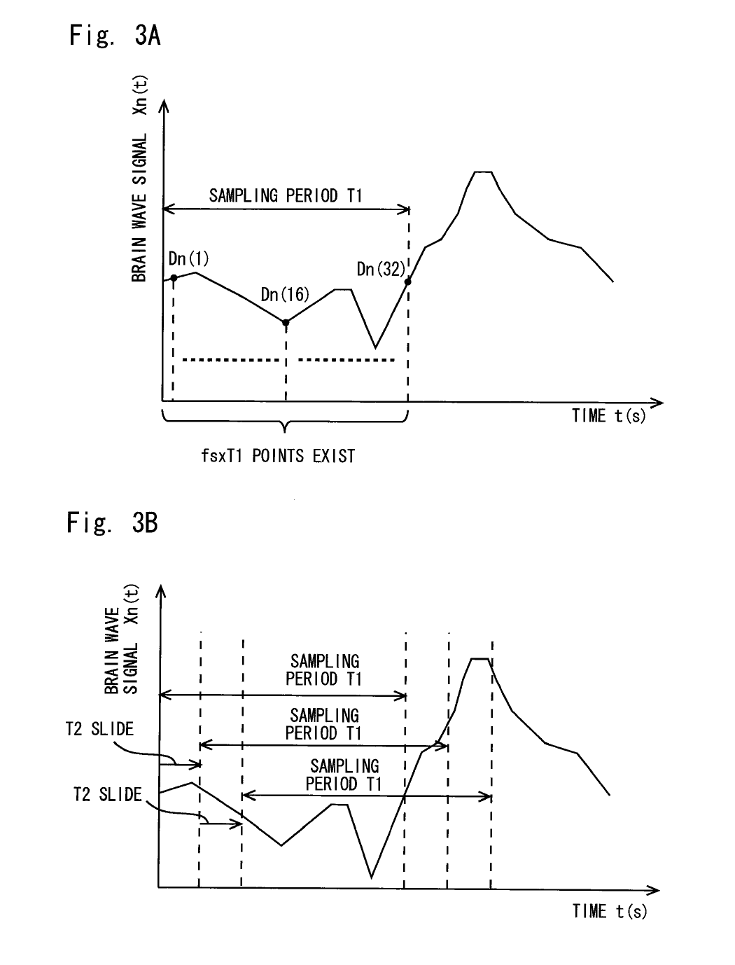

As shown in FIG. 3A, the control signal generating unit 4 first sets a frame corresponding to an interval T1 in each of brain wave signals Xn(t) (n=1 to 5), and calculates values (brain data) Dn(1) to Dn(fs×T1) of each brain wave signal at fs×T1 (fs: sampling frequency) points within the interval T1. As described above, the control signal generating unit 4 extracts brain data D1(1) to Dl(fs×T1), D2(1) to D2(fs×T1), D3(1) to D3(fs×T1), D4(1) to D4(fs×T1), and D5(1) to D5(fs×T1) from the signals X1(t), X2(t), X3(t), X4(t), and X5(t) of the electrodes 1a to 1e of the electroencephalograph 1, respectively. Then, the control signal generating unit 4 generates a matrix E of 5 (the number of electrodes)×fs×T1 (the number of brain data items within the sampling period T1) based on the extracted brain data.

$$E = \overbrace{[ \begin{matrix}D 1 ( 1 ) & D 2 ( 1 ) & D 3 ( 1 ) & D 4 ( 1 ) & D 5 ( 1 ) \\ D 1 ( 2 ) & D 2 ( 2 ) & D 3 ( 2 ) & D 4 ( 2 ) & D 5 ( 2 ) \\ ⋮ & ⋮ & ⋮ & ⋮ & ⋮ \\ D 1 ( fs \times T 1 ) & D 2 ( fs \times T 1 ) & D 3 ( fs \times T 1 ) & D 4 ( fs \times T 1 ) & D 5 ( fs \times 1 )\end{matrix} ]}^{5 Lines} \} ( fs \times T 1 ) Lines$$

Furthermore, the control signal generating unit 4 calculates the feature value fp by the following equation (1) based on the generated matrix E and filters W1 and W2 which are obtained by the well-known CSP method.

$$fp = \frac{var ( Zp )}{{\sum }_{i = 1}^{2} var ( Zt )} = \frac{var ( WpE )}{{\sum }_{i = 1}^{2} var ( WiE )} \qquad Formula ( 1 )$$

Note that “var (Zp)” mentioned above represents a variance of a data sequence Zp.

Further, the control signal generating unit 4 successively calculates the feature value fp at each predetermined short time T2 (e.g., T2=125 ms). Specifically, as shown in FIG. 3B, the control signal generating unit 4 successively generates the matrix E by sliding the sampling period T1 in each of the brain wave signals Xn(t) at each predetermined short time (predetermined interval) T2 in an overlapped manner, and successively calculates the feature value fp.

The control signal generating unit 4 performs signal processing with a learning function such as a linear SVM (Support Vector Machine) based on the calculated feature value fp, and generates control signals. Further, the control signal generating unit 4 performs learning in advance based on the linear SVM using the teacher signal received from the teacher signal generating unit 2 at the time of initial setting, and calculates a correlation between the feature value fp and the control signal (e.g., a forward movement signal, a backward movement signal, a right turn signal, a left turn signal, an acceleration signal, a deceleration signal, or a stop signal).

Note that when the accuracy of the correlation deteriorates due to changes in the brain activity state of the user, for example, the control signal generating unit 4 may execute learning based on the linear SVM again in an online state, automatically or through a user's operation. The control signal generating unit 4 successively generates control signals corresponding to the feature value fp based on the calculated feature value fp and the correlation between the feature value fp and the control signals, and sequentially outputs the generated control signals to each of the drive control unit 5 and the visual-feedback unit 6.

The drive control unit 5 sequentially executes the drive control of the mobile body 11 in response to the successive control signals from the control signal generating unit 4. For instance, the drive control unit 5 executes a forward movement control, a backward movement control, a right turn control, a left turn control, an acceleration control, a deceleration control, and a stop control of the mobile body 11 in response to a forward movement signal, a backward movement signal, a right turn signal, a left turn signal, an acceleration signal, a deceleration signal, and a stop signal, respectively, from the control signal generating unit 4.

The drive control unit 5 controls right and left motors for driving right and left drive wheels of the electric wheelchair, for example, thereby making it possible to execute the forward movement control, the backward movement control, the right turn control, the left turn control, the acceleration control, the deceleration control, and the stop control of the electric wheelchair.

In this manner, the drive control unit 5 executes the drive control of the mobile body 11 in real time in response to the control signals successively output in a short period of time from the control signal generating unit 4. This enables a highly accurate and smooth drive control of the mobile body 11 at high speed.

Further, the visual-feedback unit 6 visually presents a control result to the user according to the control signals successively sent from the control signal generating unit 4. The visual-feedback unit 6 depicts a right turn, a left turn, an acceleration, and a deceleration by using a right arrow, a left arrow, an up arrow, and a down arrow, respectively, for example.

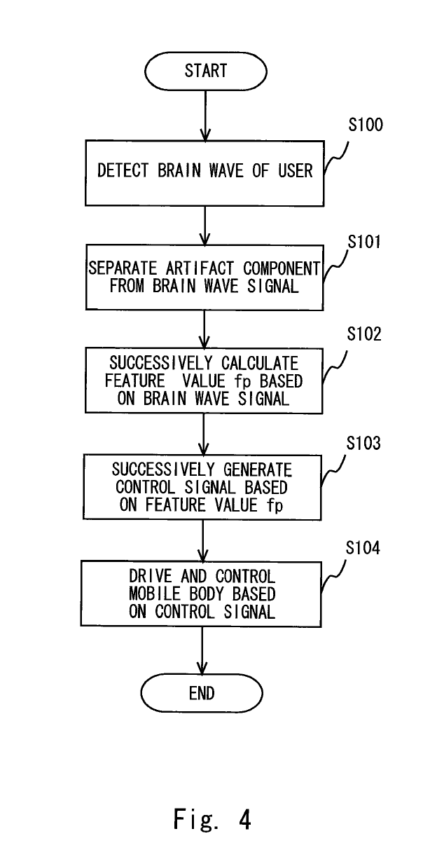

Next, a movable body control method by the mobile body control device 10 according to the first embodiment will be described in detail. FIG. 4 is a flowchart showing an example of a control processing flow of the mobile body control device according to the first embodiment.

Each of the electrodes 1a to 1e of the electroencephalograph 1 detects brain waves of the user (brain activity detection step) (step S100), and outputs the detected brain wave signals of the user to the brain signal separating unit 3.

Next, the brain signal separating unit 3 executes filter adaptive processing for separating and removing artifact components from the brain wave signals received from each of the electrodes 1a to 1e of the electroencephalograph 1 by using a blind signal separation algorithm (brain signal separation step) (step S101). The brain signal separating unit 3 outputs the brain wave signals, from which artifact components are separated, to the control signal generating unit 4.

After that, the control signal generating unit 4 successively calculates the feature value fp (p=1, 2) by spatial filtering based on the brain wave signals composed of short time sequences (feature value calculation step) (step S102). Then, the control signal generating unit 4 performs discrimination based on the calculated feature value fp, successively generates control signals (control signal generation step) (step S103), and sequentially outputs the generated control signals to the drive control unit 5.

The drive control unit 5 carries out drive control of the mobile body 11 in response to the successive control signals from the control signal generating unit 4 (drive control step) (step S104).

As described above, in the mobile body control device 10 according to the first embodiment, the control signal generating unit 4 successively generates the matrix E by sliding the sampling period T1 in each of the brain wave signals Xn(t) at each predetermined short time T2 in an overlapped manner, and successively calculates the feature value fp. Then, the drive control unit 5 executes the drive control of the mobile body 11 in real time in response to the control signals successively output in a short period of time from the control signal generating unit 4. Thus, the control and discrimination in the brain wave signals, which are segmented at each sampling period T1, are successively repeated at each predetermined short time T2 in an overlapped manner. Based on the combined results of control and discrimination, which are successively obtained in a short period of time, the macroscopic operation of the mobile body 11 can be controlled. Consequently, a highly accurate and smooth drive control of the mobile body 11 can be achieved at high speed (in real time).

Moreover, as described above, a highly accurate and smooth drive control of the mobile body 11 can be achieved at high speed by a simple control processing in which the feature value fp is successively calculated by sliding the sampling period T1 in each of the brain wave signals Xn(t) at each predetermined short time T2 in an overlapped manner, to thereby generate the corresponding control signals. That is, a highly accurate control can be achieved while simplifying the control processing in the mobile body control device 10 and the mobile body control method.

### Second Embodiment

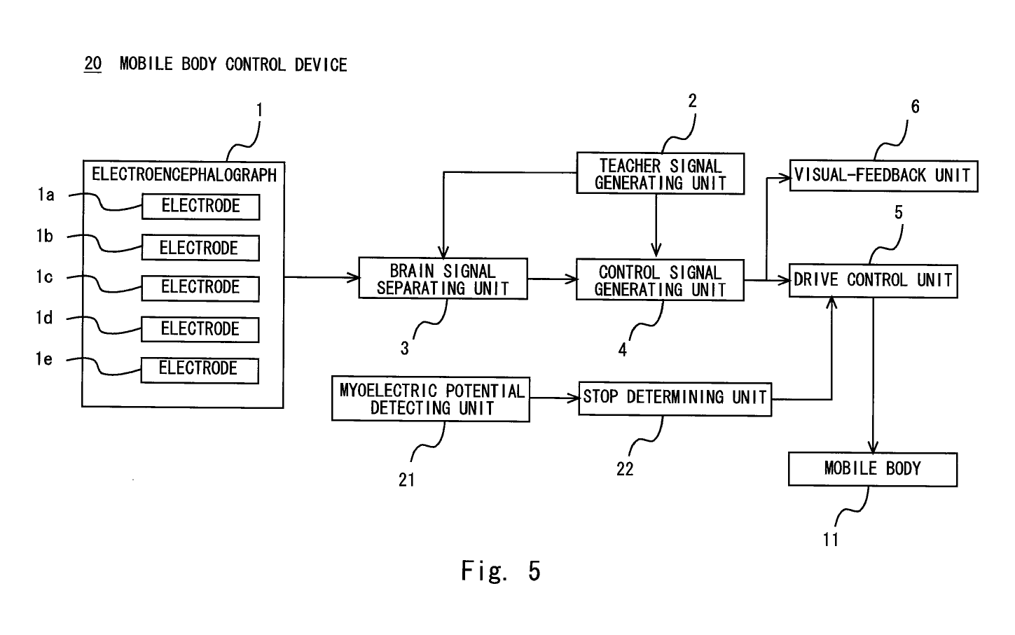

FIG. 5 is a block diagram showing an example of a system configuration of a mobile body control device according to a second embodiment of the present invention. A mobile body control device 20 according to the second embodiment includes a myoelectric potential detecting unit 21 and a stop determining unit 22 in addition to the components of the mobile body control device 10 according to the first embodiment.

The myoelectric potential detecting unit 21 includes one or more myoelectric sensors such as a dry surface electrode, a wet surface electrode, or a silver/silver chloride plate electrode. The myoelectric sensors are attached to cheek or neck areas or the like which can instantly be moved by the user. This makes it possible to easily and reliably detect the myoelectric potential of the user. Each of the myoelectric sensors of the myoelectric potential detecting unit 21 outputs the detected myoelectric potential to the stop determining unit 22 as a myoelectric potential signal.

The stop determining unit 22 determines whether or not to stop the mobile body 11 based on the myoelectric potential signal from the myoelectric potential detecting unit 21. When determining that the mobile body 11 is to be stopped, the stop determining unit 22 outputs a stop signal to the drive control unit 5. Upon receiving the stop signal from the stop determining unit 22, the drive control unit 5 executes a stop control of the mobile body 11.

For instance, when the myoelectric potential signal output from the myoelectric sensor of the myoelectric potential detecting unit 21 is equal to or greater than a predetermined value and when the stop determining unit 22 determines the motion of the cheek (or the neck, etc.) as a motion indicative of an emergency state of the user, the stop determining unit 22 outputs the stop signal to the drive control unit 5. Upon receiving the stop signal from the stop determining unit 22, the drive control unit 5 executes the stop control to bring the mobile body 11 to an emergency stop.

The other components of the mobile body control device 20 according to the second embodiment are substantially the same as those of the mobile body control device 10 according to the first embodiment. Accordingly, in the mobile body control device 20 according to the second embodiment, identical components are denoted by identical reference numerals, and the detailed description thereof is omitted.

As described above, in the mobile body control device 20 according to the second embodiment, when determining that the movably body 11 is to be stopped based on the myoelectric potential signal from the myoelectric sensor of the myoelectric potential detecting unit 21, the stop determining unit 22 outputs the stop signal to the drive control unit 5. Then, upon receiving the stop signal from the stop determining unit 22, the drive control unit 5 executes the stop control of the mobile body 11. This makes it possible to reliably stop the mobile body 11 in response to a natural reaction of the user when the user brings the mobile body 11 to a stop.

### Third Embodiment

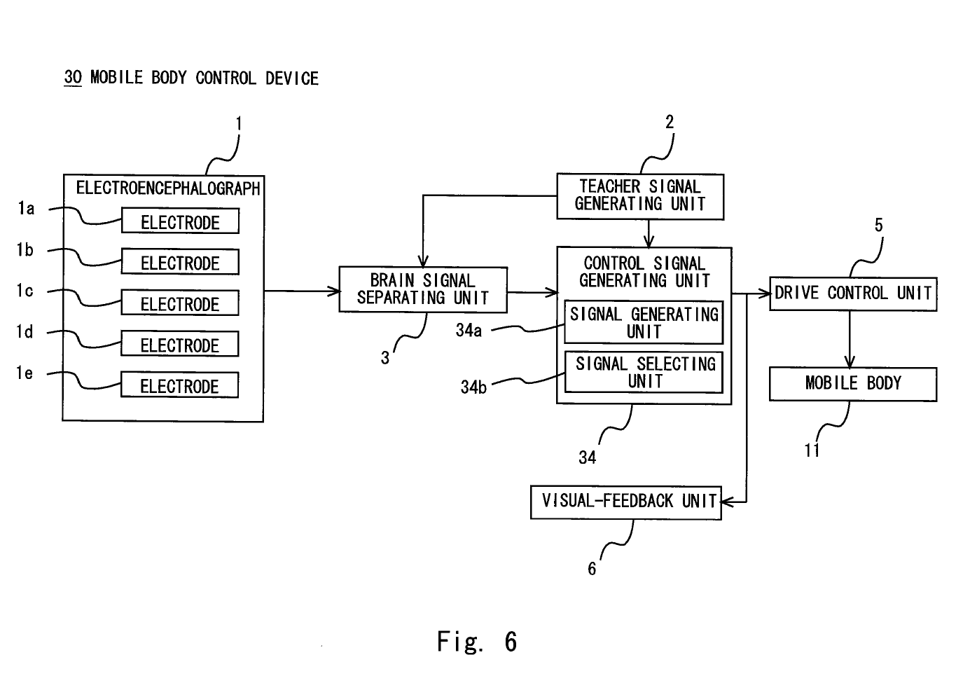

FIG. 6 is a block diagram showing an example of a system configuration of a mobile body control device according to a third embodiment of the present invention. In a mobile body control device 30 according to the third embodiment, a control signal generating unit 34 includes a signal generating unit 34a which successively generates control signals corresponding to the feature value fp based on the calculated feature value fp and the correlation between the feature value fp and the control signal, and a signal selecting unit 34b which divides the control signals successively generated by the signal generating unit 34a into groups each having a predetermined number of (e.g., three) successive control signals, and selects, for each group, a control signal of a type which is largest in number in each of the groups.

Herein, the signal selecting unit 34b forms groups each having a current control signal and successive previous control signals. For instance, the signal selecting unit 34b selects the current control signal, the previous control signal, and the last-but-one control signal as one group. However, the control signals to be selected are not limited thereto. For instance, the current control signal, the previous control signal, the last-but-one control signal, and the last-but-two control signal may be selected as one group. Any group configuration may be applied. Note that as the number of control signals constituting one group is increased, the accuracy of the control signals to be generated is increased, which enables more stable operation of the mobile body 11. Further, the signal selecting unit 34b selects a single control signal from each group, but the number of control signals to be selected is not limited thereto. A plurality of control signals may be selected. The signal selecting unit 34b sequentially outputs the selected control signal to each of the drive control unit 5 and the visual-feedback unit 6.

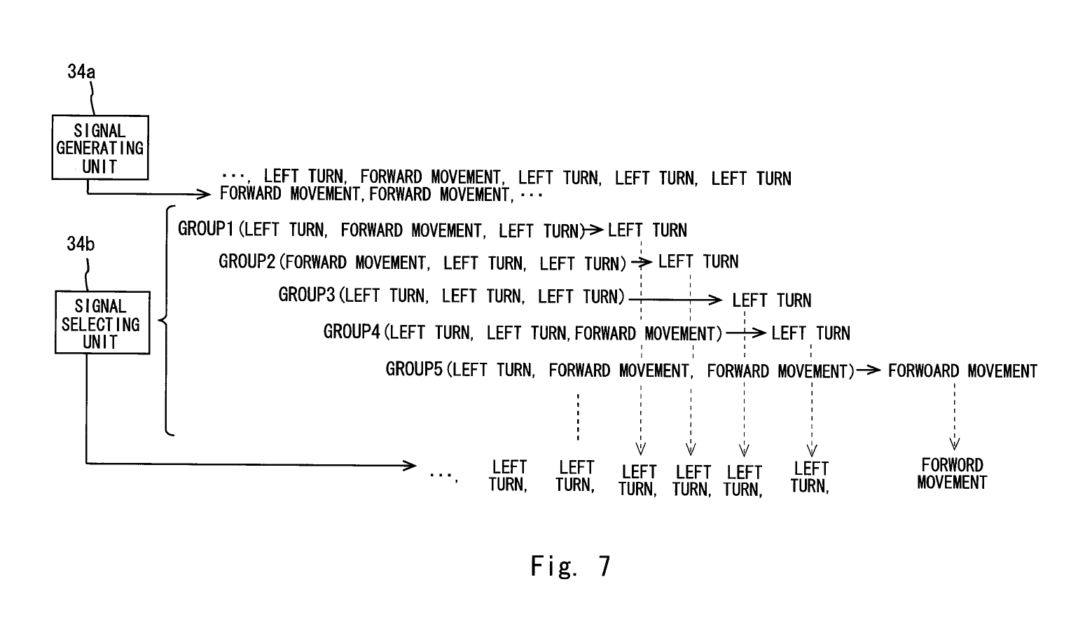

For instance, assume that the signal generating unit 34a successively generates control signals for “left turn, forward movement, left turn, left turn, left turn, forward movement, and forward movement”. In this case, as shown in FIG. 7, the signal selecting unit 34b divides the successive control signals generated by the signal generating unit 34a into groups of Group 1 (left turn, forward movement, left turn), Group 2 (forward movement, left turn, left turn), Group 3 (left turn, left turn, left turn), Group 4 (left turn, left turn, forward movement), Group 5 (left turn, forward movement, forward movement), . . . . Then, the signal selecting unit 34b selects, for each group, a control signal of a type which is largest in number in each group, i.e., “left turn, left turn, left turn, left turn, forward movement”, and sequentially outputs the selected output signal to each of the drive control unit 5 and the visual-feedback unit 6.

In this manner, the mobile body control device 30 according to the third embodiment can improve the accuracy of the control signals generated by the control signal generating unit 34, and can allow the mobile body 11 to operate stably. For instance, in some cases, an inexperienced operator of the mobile body 11 cannot accurately imagine the operation of the mobile body 11, and the brain wave signals may be disturbed. Also in such a case, the mobile body control device 30 according to the third embodiment corrects the disturbance of the brain waves, thereby allowing the operator to operate the mobile body 11 more accurately. Note that the disturbance of the brain signals indicating operation different from the use's true intention may appear instantly before switching of the operation, for example. Therefore, it is effective to determine the control content based on majority logic. However, the signal selecting unit 34b described above may select the control signal based on time average, medians, or statistics, for example, instead of selecting the control signal of the type which is largest in number in each group, based on the majority logic.

Note that the other components of the mobile body control device 30 according to the third embodiment are substantially the same as those of the mobile body control device 10 according to the first embodiment. Accordingly, in the mobile body control device 30 according to the third embodiment, identical components are denoted by identical reference numerals, and the detailed description thereof is omitted.

### Fourth Embodiment

The above-mentioned second embodiment illustrates an example in which the stop control of the mobile body 11 is carried out using the detection result of the myoelectric potential. However, the utilization of the myoelectric potential is not limited to the stop control. In this embodiment, a specific example is described in which control of the mobile body 11 based on the brain activity information is integrated with control of the mobile body 11 based on the, myoelectric potential.

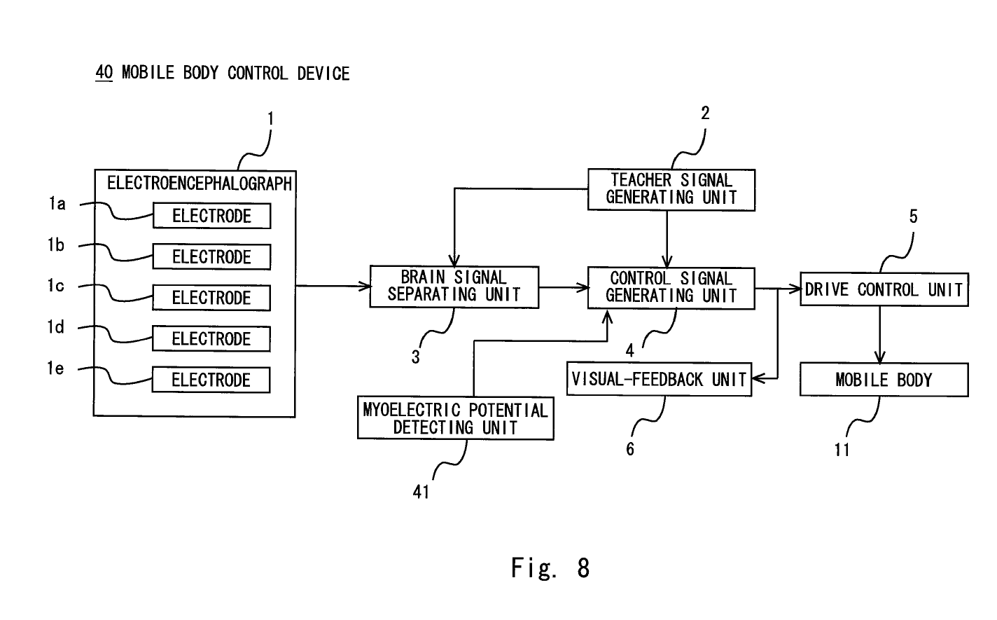

FIG. 8 is a block diagram showing an example of a system configuration of a mobile body control device 40 according to a fourth embodiment of the present invention. The mobile body control device 40 according to the fourth embodiment includes a myoelectric potential detecting unit 41 in addition to the components of the mobile body control device 10 according to the first embodiment. The myoelectric potential detecting unit 41 generates a myoelectric potential signal indicating a fluctuation in the myoelectric potential of the user, in the same manner as the myoelectric potential detecting unit 21 described above. Note that the myoelectric potential detecting unit 41 may have the same configuration as the myoelectric potential detecting unit 21. The generated myoelectric potential signal is supplied to the control signal generating unit 4.

The control signal generating unit 4 according to this embodiment generates a control signal to be supplied to the drive signal generating unit 5, by compositely using the brain wave signal supplied from the brain signal separating unit 3 and the myoelectric potential signal supplied from the myoelectric potential detecting unit 41. Specifically, the control signal generating unit 4 compares a control content based on the brain wave signal, i.e., the brain activity information, with a control content based on the myoelectric potential signal, i.e., the myoelectric potential. Then, when these contents conflict with each other, the control signal generating unit 4 generates a control signal indicating the control content based on the myoelectric potential. In other words, the operation of the mobile body 11 is controlled by preferentially using the myoelectric potential over the brain activity information. For example, when the brain activity information indicates forward movement and the myoelectric potential indicates stop, the control signal generating unit 4 generates a control signal for causing the mobile body 11 to perform a stop operation. Further, for example, when the brain activity information indicates direct advance and the myoelectric potential indicates left or right turn, the control signal generating unit 4 generates a control signal for causing the mobile body 11 to perform a turning operation.

According to this embodiment, the mobile body 11 can be driven and controlled more safely. Note that in the mobile body control device 40 according to this embodiment, the other components are substantially the same as those of the mobile body control device 10 according to the first embodiment. Accordingly, in the mobile body control device 40 according to the fourth embodiment, identical components are denoted by identical reference numerals, and the detailed description thereof is omitted.

### Fifth Embodiment

The above-mentioned second embodiment illustrates an example of sampling the output of brain wave signals obtained by the electroencephalograph, and performing majority evaluation in a time direction for control contents represented by each sample. In this embodiment, a plurality of sensors (e.g., electrodes) included in the electroencephalograph are divided into three or more sensor groups (e.g., electrode groups), and when the control contents represented by the outputs of each sensor group at the same time are different from each other, one control content is selected by majority evaluation.

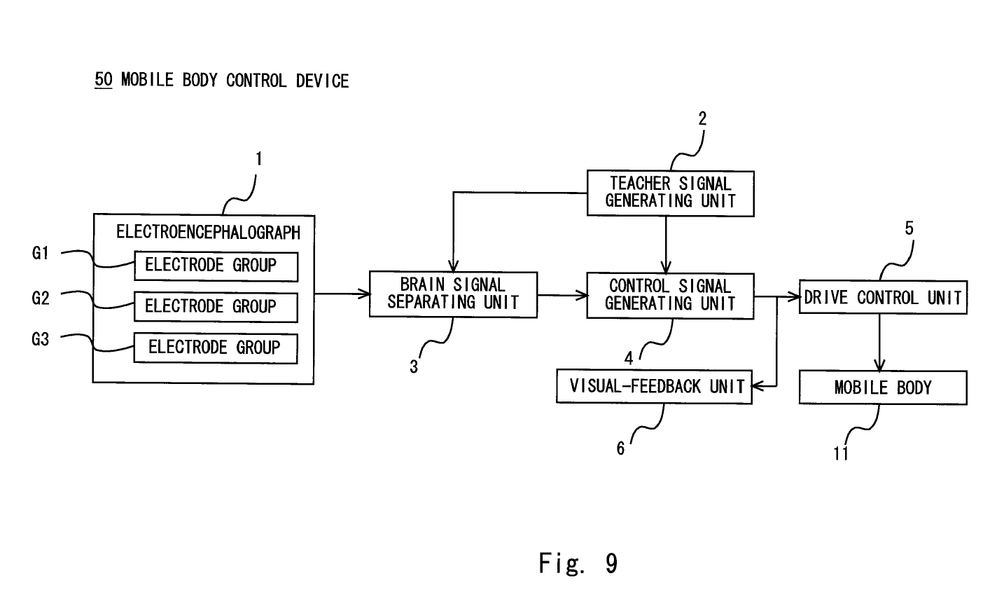

FIG. 9 is a block diagram showing an example of a system configuration of a mobile body control device 50 according to a fifth embodiment of the present invention. The electrodes included in the electroencephalograph 1 of this embodiment are divided into a plurality of electrode groups. One electrode group includes at least one electrode. In the example of FIG. 9, the electroencephalograph 1 includes three electrode groups G1 to G3.

The brain signal separating unit 3 performs digital sampling of an analog brain wave signal supplied from each electrode included in the electrode groups G1 to G3, and performs adaptive filtering processing using a blind signal separation algorithm, thereby generating a brain wave signal group from which artifact components are removed. Note that the signal processing in the brain signal separating unit 3 may be similar to that of the first embodiment described above.

The control signal generating unit 4 receives the brain wave signal group, from which the artifact components are removed, from the brain signal separating unit 3, and determines a control content (e.g., forward movement control, backward movement control, right turn control, or left turn control) for the mobile body 11 for each of the electrode groups G1 to G3 by using the brain wave signals in each of the electrode groups G1 to G3. Then, when the control contents in each of the electrode groups G1 to G3 are different from each other, one control content is determined by majority evaluation. For instance, when the control content determined based on the outputs of the electrode groups G1 and G2 indicates “left turn” and the control content determined based on the output of the electrode group G3 indicates “direct advance”, the control signal generating unit 4 supplies a control signal indicating “left turn” to the drive control unit

In this manner, the control content for the mobile body 11 is determined by majority evaluation among the plurality of sensor groups, thereby making it possible to further suppress the occurrence of malfunction of the mobile body 11 due to the disturbance of the brain waves.

Although the best modes for carrying out the present invention have been described above by way of embodiments, the present invention is not particularly limited to the above embodiments. Various modifications or alternations can be made to the above embodiments without departing from the scope of the present invention.

In the first to fifth embodiments, the brain activity information of the user is detected by the electroencephalograph 1, but the detection method is not limited thereto. For instance, the brain activity information of the user may be detected by NIRS (Near Infrared Spectroscopy) using near-infrared light. In other words, the electroencephalograph 1 is merely an example of the brain activity detecting unit. As the brain activity detecting unit, any electroencephalograph capable of detecting the brain activity information of the user can be applied. Furthermore, the brain waves of the user are used as the brain activity information, but the brain activity information is not limited thereto. For example, any brain information such as information about an oxygenated hemoglobin state in cerebral blood can be applied.

In the first to fourth embodiments, the electroencephalograph 1 includes the five electrodes 1a to 1e, but the configuration of the electroencephalograph 1 is not limited thereto. For example, one or any number of electrodes may be included, and the attachment position thereof on the head is also arbitrary.

In the first to fifth embodiments, the brain signal separating unit 3 separates artifact components from each of the brain wave signals by using the blind signal separation algorithm, but the signal separation algorithm is not limited thereto. Any signal separation algorithm can be used as long as the artifact components can be appropriately separated from each of the brain wave signals.

In the first to fifth embodiments, the drive control unit 5 may have a limiter function in which, only when a same control signal is successively received from the control signal generating unit 4 a predetermined number of times or more, the drive control of the mobile body 11 corresponding to the control signal is executed. This enables highly accurate drive control of the mobile body 11.

In the first to fifth embodiments, an electric wheelchair is applied as the mobile body 11, but the application of the present invention is not limited thereto. The present invention can be applied to any mobile apparatus that travels with a user riding thereon.

Furthermore, the present invention can also be applied to robots other than mobile apparatus, a cursor on a PC, or the like, as a control target.

In the first to fifth embodiments, the control signal generating units 4 and 34 calculate the feature value fp by the aforementioned equation (1) based on the generated matrix “E”, but the calculation method is not limited thereto. The feature value for the brain wave signals may be calculated by any calculation method.

Further, in the first to fifth embodiments, the visual-feedback unit 6 visually presents the control result to a user in response to the successive control signals from the control signal generating unit 4, but the method is not limited thereto. For instance, the control result may be presented to the user auditorily using sound or the like, tactually using vibration or the like, or by a combination thereof. Any perception method for allowing a user to perceive the control result may be employed. Furthermore, the first to fifth embodiments may be combined as necessary.

This application is based upon and claims the benefit of priority from Japanese patent application No. 2008-278348, filed on Oct. 29, 2008, the disclosure of which is incorporated herein in its entirety by reference.

### Reference Signs List

1 ELECTROENCEPHALOGRAPH

1a, 1b, 1c, 1d, 1e ELECTRODE

2 TEACHER SIGNAL GENERATING UNIT

3 BRAIN SIGNAL SEPARATING UNIT

4, 34 CONTROL SIGNAL GENERATING UNIT

5 DRIVE CONTROL UNIT

6 VISUAL-FEEDBACK UNIT

10, 20, 30, 40, 50 MOBILE BODY CONTROL DEVICE

11 MOBILE BODY

21, 41 MYOELECTRIC POTENTIAL DETECTING UNIT

22 STOP DETERMINING UNIT

34a SIGNAL GENERATING UNIT

34b SIGNAL SELECTING UNIT

G1, G2, G3 ELECTRODE GROUP

1. A mobile body control device comprising:
a brain activity detecting unit that detects brain activity information of a user;
a brain signal separating unit that separates an artifact component from the brain activity information detected by the brain activity detecting unit;
a control signal generating unit for sliding a sampling period for extracting brain data, at predetermined intervals in an overlapped manner in the brain activity information from which the artifact component is separated by the brain signal separating unit, successively calculating feature values for the brain data within each of the sampling periods obtained by sliding, and generating a control signal based on the feature values calculated; and
a drive control unit that drives and controls a mobile body with a user riding thereon, based on the control signal generated by the control signal generating unit.

2. The mobile body control device according to claim 1, further comprising a teacher signal generating unit that generates a teacher signal,
wherein the brain signal separating unit performs learning using the teacher signal generated by the teacher signal generating unit, and separates the artifact component depending on a user.

3. The mobile body control device according to claim 1, further comprising a teacher signal generating unit that generates a teacher signal,
wherein the control signal generating unit calculates a correlation between the feature values and the control signal by using the teacher signal generated by the teacher signal generating unit, and generates the control signal based on the feature values and the correlation calculated.

4. The mobile body control device according to claim 1, further comprising:
a myoelectric potential detecting unit that detects a myoelectric potential of a user; and
a stop determining unit that determines whether or not to stop the mobile body based on the myoelectric potential detected by the myoelectric potential detecting unit,
wherein when the stop determining unit determines to stop the mobile body, the drive control unit controls the mobile body to be stopped.

5. The mobile body control device according to claim 1, wherein when the drive control unit successively receives a same control signal from the control signal generating unit a predetermined number of times or more, the drive control unit executes a control corresponding to the control signal.

6. The mobile body control device according to claim 1, wherein
the brain activity detecting unit includes a plurality of sensors that detect a brain wave signal of a user, and
the control signal generating unit slides a sampling period for extracting brain data, at predetermined intervals in an overlapped manner in a plurality of the brain wave signals which are detected by the plurality of sensors and from which the artifact component is separated, successively calculates feature values for the brain data within each of the sampling periods obtained by sliding, and generates a plurality of control signals based on the plurality of feature values calculated.

7. The mobile body control device according to claim 1, wherein the control signal generating unit comprises:
a signal generating unit that successively generates control signals corresponding to the feature values based on the feature values calculated and on a preset correlation between the feature values and the control signals; and
a signal selecting unit that divides the control signals successively generated by the signal generating unit into groups each having a predetermined number of successive control signals, selects at least one control signal from each of the groups, and outputs the selected control signal to the drive control unit.

8. The mobile body control device according to claim 7, wherein the signal selecting unit selects, for each of the groups, a control signal of a type which is largest in number in each of the groups, and outputs the selected control signal to the drive control unit.

9. The mobile body control device according to claim 7, wherein the signal selecting unit forms the groups each having a current control signal and successive previous control signals.

10. The mobile body control device according to claim 1, further comprising a myoelectric potential detecting unit that detects a myoelectric potential of a user, wherein
the control signal generating unit is capable of generating the control signal based on the myoelectric potential detected by the myoelectric potential detecting unit, and
when a control content based on the brain activity information is different from a control content based on the myoelectric potential, the control signal generating unit supplies the control signal indicating the control content based on the myoelectric potential to the drive control unit.

11. The mobile body control device according to claim 1, wherein the control signal generating unit sequentially makes judgment using the feature values, determines one control content by majority among a plurality of results of judgment sequentially made, and supplies the control signal indicating the control content determined by the majority to the drive control unit.

12. The mobile body control device according to claim 1, wherein
the brain activity detecting unit includes at least three sensor groups,
each of the at least three sensor groups includes at least one sensor that detects a brain wave signal of a user, and
the control signal generating unit determines one control content by majority when control contents based on the brain activity information detected by each of the sensor groups are different from each other, and supplies the control signal indicating the control content determined by the majority to the drive control unit.

13. The mobile body control device according to claim 1, further comprising a perception unit for allowing a user to perceive a control result based on the control signal generated by the control signal generating unit.

14. The mobile body control device according to claim 13, wherein the perception unit is a visual-feedback unit that visualizes the control result.

15. A mobile body control method comprising:
detecting brain activity information of a user;
separating an artifact component from the brain activity information detected;
sliding a sampling period for extracting brain data, at predetermined intervals in an overlapped manner in the brain activity information from which the artifact component is separated, and successively calculating feature values for the brain data within each of the sampling periods obtained by sliding;
generating a control signal based on the feature values calculated; and
driving and controlling a mobile body with the user riding thereon, based on the control signal generated.

16. A mobile body control device comprising:
brain activity detecting means for detecting brain activity information of a user;
brain signal separating means for separating an artifact component from the brain activity information detected by the brain activity detecting means;
control signal generating means for sliding a sampling period for extracting brain data, at predetermined intervals in an overlapped manner in the brain activity information from which the artifact component is separated by the brain signal separating means, successively calculating feature values for the brain data within each of the sampling periods obtained by sliding, and generating a control signal based on the feature values calculated; and
drive control means for driving and controlling a mobile body with a user riding thereon, based on the control signal generated by the control signal generating means.
